(19) United States
(12) Patent Application Publication
(10) Pub. No.: US 2020/0199645 A1
(43) Pub. Date: Jun. 25, 2020

(54) DEVICE AND METHOD FOR DIAGNOSIS OF ALZHEIMER'S SYMPTOMS

(71) Applicants: Control of Innate Immunity Technology Research Association, Takamatsu-shi (JP); Hamamatsu Photonics K.K., Hamamatsu-shi (JP); National University Corporation Kagawa University, Takamatsu-shi (JP)

(72) Inventors: Gen-Ichiro Soma, Tokyo (JP); Hiroyuki Inagawa, Takamatsu-shi (JP); Kimiko Kazumura, Hamamatsu-shi (JP); Yutaro Kobayashi, Kita-gun (JP)

(21) Appl. No.: 16/643,695

(22) PCT Filed: Aug. 27, 2018
(86) PCT No.: PCT/JP2018/031554

§ 371 Date: Mar. 2, 2020

(30) Foreign Application Priority Data
Sep. 8, 2017 (JP) 2017-173037

(51) Int. Cl. C12Q 1/28; C12Q 1/06; G01N 33/49

(52) U.S. Cl. CPC C12Q 1/28; G01N 2800/2814; G01N 33/49; C12Q 1/06

(57) ABSTRACT

To provide a pathological index for Alzheimer's disease conveniently and with high precision, an Alzheimer's disease diagnosis device that includes a measurement means configured to measure one index or more selected from the group consisting of superoxide production activity, myeloperoxidase activity, oxidized LDL level, phagocytosis, triglycerides, fasting blood glucose, total cholesterol, hemoglobin A1c, and insulin in peripheral blood and a displaying means configured to display an index measured by the measurement means as a pathological index for Alzheimer's disease.

### TECHNICAL FIELD

The present invention relates to a device and method for diagnosis of Alzheimer's symptoms using a neutrophil function evaluation system, etc.

### BACKGROUND ART

In recent years in Japan, the number of dementia patients has been increasing every year with the aging of the population. The number of patients in Japan has currently exceeded 4.60 million people and is estimated to reach 7 million people or one in five aged persons in 2025. Of the dementia patients, approximately 60% suffer from Alzheimer's disease, approximately 20% suffer from vascular dementia, and the remainder include patients of various dementing disorders such as Lewy body dementia, etc. The cause, treatment method, and prevention method of Alzheimer's disease are yet unclear and medical solutions are needed urgently. With the diagnostic criteria for Alzheimer's disease proposed in 2011 by the NIA/AA (The National Institute on Aging and the Alzheimer's Association), Alzheimer's disease is classified according to the three stages of preclinical stage, mild cognitive impairment (MCI), and Alzheimer's disease dementia and major clinical diagnostic criteria and research diagnostic criteria are presented. The former are clinical findings on cognitive impairment (memory disorders, aphasia, apraxia, etc.), mental disorders (depression, insomnia, hallucinations, etc.), etc. Although the latter include biomarker evaluations related to Alzheimer's disease (quantification of amyloid R and tau proteins in cerebrospinal fluid), imaging of cerebral amyloid accumulation by PET (positron emission tomography), evaluation of cerebral atrophy by MRI, etc., relationships with pathological changes have not been elucidated sufficiently for many of such diagnostic markers and issues of high invasiveness and expensive device and inspection costs, etc., remain. Biochemical diagnostic markers that enable detection of onset of Alzheimer's disease in a convenient and low invasive manner are thus considered to be especially effective for performing early diagnosis and preclinical diagnosis of Alzheimer's disease. So far, measurements of biochemical markers in blood, such as various inflammatory cytokines, oxidative stress markers (for example, lipid peroxides, 4-hydroxy-2-nonenal (4-HNE), advanced glycation end products (AGEs)), micro RNAs, etc., as diagnostic markers for Alzheimer's disease have been proposed (for example, NPL 1). In recent years, oxidative stress of peripheral blood has been indicated to be involved in initial stages of Alzheimer's disease (NPL 2).

### CITATION LIST

### Patent Literature

[PTL 1] JP 2015-084757 A
[PTL 2] JP 2017-074008 A
[PTL 3] JP 2017-040473 A

### Non Patent Literature

[NPL 1] N. Sharma et al., Journal of Clinical and Diagnostic Research, 10, 1-6, 2016
[NPL 2] M. Schrag et al., Neurobiology of Disease, 59, 100-110, 2013

### SUMMARY OF INVENTION

### Technical Problem

Neutrophils are immunocompetent cells that are involved in biological defense and upon recognizing a xenobiotic, uses the enzyme, NADPH (nicotinamide adenine dinucleotide phosphate) oxidase, to produce a superoxide anion radical (so-called superoxide; O₂^{⋅-}), which is a reactive oxygen species. Further, the enzyme myeloperoxidase (MPO) produces hypochlorous acid using hydrogen peroxide, which is a superoxide metabolite, as a substrate. Such reactive oxygen species, although controlling various in vivo responses (for example, cell cycle and phagocytic response) at physiological concentrations, induce inflammatory responses in tissue when produced excessively and it is thus indicated that neutrophil activity, etc., at particular sites in the brain are involved in the onset of oxidative stress related ailments, such as Alzheimer's disease. On the other hand, if neutrophil activity, etc., in peripheral blood that are independent of the interior of the brain, which is isolated by the blood-brain barrier, are related to Alzheimer's disease, pathological indices of Alzheimer's disease can be evaluated conveniently by measuring the neutrophil activity, etc., in peripheral blood. Incidentally, Kazumura et al., have developed a method for simultaneously evaluating MPO activity and superoxide production activity in blood by a convenient procedure using a real time measurement system for fluorescence and chemiluminescence (PTL 1 and 3) and have also disclosed a method for evaluating phagocytic capacity of phagocytes, such as neutrophils, etc., (PTL 2).

Thus, an object of the present invention is to provide a device and method for diagnosis of Alzheimer's disease using a neutrophil activity evaluation system disclosed in PTL 1 to 3 (also referred to hereinafter simply as “neutrophil activity evaluation system”), etc.

### Solution to Problem

An Alzheimer's disease diagnosis device according to the present invention is characterized in including a measurement means configured to measure one index or more selected from the group consisting of superoxide production activity, myeloperoxidase activity, oxidized LDL level, phagocytosis, triglycerides, fasting blood glucose, total cholesterol, hemoglobin A1c, and insulin in peripheral blood and a displaying means configured to display an index measured by the measurement means as a pathological index for Alzheimer's disease.

Also, the measurement means can provide a pathological index for Alzheimer's disease with higher precision by measuring two indices or more selected from the group consisting of superoxide production activity, myeloperoxidase activity, oxidized LDL level, and phagocytosis and with which superoxide production activity is selected.

Also, the measurement means can provide a pathological index for Alzheimer's disease with even higher precision by measuring superoxide production activity, myeloperoxidase activity, and oxidized LDL level.

Also, an Alzheimer's disease diagnosis device according to the present invention is characterized in including a measurement means configured to measure superoxide production activity, myeloperoxidase activity, oxidized LDL level, and phagocytosis in peripheral blood and a displaying means configured to display a×A+b×B+c×C+d×D with respect to the indices measured by the measurement means as a pathological index for Alzheimer's disease.

wherein,

A: normalized superoxide production activity

B: normalized myeloperoxidase activity

C: normalized oxidized LDL level

D: normalized phagocytosis

a, b, c, d: coefficients

Also, an Alzheimer's disease diagnosis method according to the present invention is a method with which one index or more selected from the group consisting of superoxide production activity, myeloperoxidase activity, oxidized LDL level, phagocytosis, triglycerides, fasting blood glucose, total cholesterol, hemoglobin A1c, and insulin in collected peripheral blood is used as a pathological index for Alzheimer's disease.

### Advantageous Effects of Invention

By the present invention, a pathological index for Alzheimer's disease can be provided conveniently and with high precision.

### BRIEF DESCRIPTION OF DRAWINGS

FIG. 1 is a diagram showing correlation with a water maze test.

### DESCRIPTION OF EMBODIMENTS

A mode for implementing the present invention shall now be described in detail with reference to the attached drawing.

Evaluation of neutrophil activity according to the present embodiment is in accordance with the methods described in PTL 1 and PTL 3. That is, MPO activity in a sample is based on a fluorescence detection method using aminophenyl fluorescein (APF) as an indicator, and superoxide production activity is based on a chemiluminescence method using 2-methyl-6-(4-methoxyphenyl)-3,7-dihydroimidazo[1,2-a]pyrazin-3-one (MCLA) as an indicator. An inflammatory defense ability (which can also be called an antioxidant ability or oxidative stress preventing ability) of neutrophils can be evaluated by adding a neutrophil stimulant to the sample and therefore, phorbol 12-myristate 13-acetate (PMA) is used in the present embodiment. The net MPO activity or superoxide production activity in the sample due to the neutrophil stimulant can be evaluated as a value obtained by subtracting a fluorescence amount or chemiluminescence amount before stimulation respectively from a maximum fluorescence amount or chemiluminescence amount after stimulant addition. Also, phagocytosis in the sample is in accordance with the method described in PTL 2. That is, it is based on a fluorescence detection method using phagocytosed particles labeled with a pH-sensitive fluorescent dye (manufactured by Thermo Fisher Scientific Inc.) as an indicator. Oxidized LDL level in mouse peripheral blood can be evaluated using a commercially available ELISA kit (Kamiya Biomedical Company).

For experiments, 12- to 14-week-old, male SAMP8 mice (SAMP8/Ta Slc; Japan SLC, Inc.) were used as Alzheimer's disease model mice and after one week of prefeeding, the mice were divided into two groups, a high-fat diet (animal diet containing 35% fat (Research Diets, Inc.)) was given to one group, and a low-fat diet was given to the other group (details will be described later). The development of Alzheimer's disease was favored by giving the high-fat diet. Water was provided ad libitum. The mice were fed in a temperature- and humidity-controlled vivarium under environmental conditions with food and water ad libitum on a 12 h/12 h light/dark cycle. After feeding for 17 weeks, a water maze test described below was performed for one week to evaluate learning function. On the day following the end of the water maze test, blood was collected from the heart. The present animal experiments have been approved by the Kagawa University Animal Experiment Committee.

The superoxide production activity, MPO activity, and phagocytosis of leucocytes that are involved in vivo inflammatory responses were measured using a neutrophil activity evaluation system (CFL-P2200; Hamamatsu Photonics K.K.) (PTL 1 to 3). Heparin was used as anticoagulant in blood collection. Centrifugal separation (1200 g, 20 minutes) was performed on the blood to obtain plasma. The commercially available kits shown below were used for evaluation in biochemical analysis of the plasma.

Insulin (insulin): mouse insulin ELISA kit (Shibayagi)
Hemoglobin A1c (HbA1c): HbA1c measuring kit (Sekisui Medical)
Triglycerides (TG), total cholesterol (TC): corresponding measuring kits (Wako Pure Chemicals)
Fasting blood glucose (fasting BG): Blood sugar self-measuring instrument (Roche Diagnostics)

In the present example, the mice were divided into the following two groups.

(1) NC group: The animal diet containing 4% fat (low-fat animal diet) and water were provided ad libitum.
(2) PC group: The animal diet containing 35% fat (high-fat animal diet) and water were provided ad libitum.

[Superoxide (O₂^{⋅-}) Production Activity] and [Myeloperoxidase (MPO) Activity]

The neutrophil activity (O₂^{⋅-} production activity and MPO activity) of mouse peripheral blood was evaluated using a prototype neutrophil activity evaluation device (PTL 1 and 3). 500 μL of a hemolytic reagent (Tonbo Biosciences) were added to 30 μL of mouse peripheral blood and after letting react for 2 minutes at room temperature, centrifugal separation at 200×g was performed for 3 minutes and a cell suspension was recovered. As the hemolytic reagent, commercially available one may be used, but one without a cell immobilizing agent is preferable. To the neutrophil fraction obtained from the 30 μL of blood, a chemiluminescent reagent (MCLA; final concentration: 0.5 μM) and a fluorescent reagent (APF; final concentration: 2 μM) were added and a buffer solution (154 mM sodium chloride, 5.6 mM potassium chloride, 10 mM HEPES, and 1 mM calcium chloride) was used to achieve a total volume of 500 μL. The measurement sample was set in the prototype neutrophil activity evaluation device and chemiluminescence and fluorescence values before and after stimulation by PMA (final concentration: 1 μM) were measured in real time (every 0.5 seconds). The values of superoxide production activity and MPO activity were set to the differences in measured fluorescent intensity values before and after PMA stimulation. The respective measured values were converted such that the average value becomes 0 and the standard deviation becomes 1 (normalization (Wikipedia: An operation by which numerical quantities are made dimensionless quantities by dividing by a representative value, etc., such as to enable comparison with each other is called normalization. For multivariate analysis an operation of ‘linearly converting such that the average becomes 0 and the dispersion becomes 1” is used.)).

[Oxidized LDL (oxLDL)]

The oxidized LDL level in mouse peripheral blood was measured using the commercially available ELISA kit (Kamiya Biomedical Company). As the measurement method, a protocol provided with the kit was followed and a sample prepared by diluting the mouse plasma by 1000 times with a buffer solution provided with the kit was subject to measurement. Respective measured values were converted (normalized) such that the average value becomes 0 and the standard deviation becomes 1.

[Phagocytic Capacity (Phagocytosis)]

The phagocytosis of mouse peripheral blood was evaluated using a phagocyte phagocytic capacity evaluation apparatus (PTL 2). For measurement, pH-sensitive fluorescent particles (Green E. coli) were added to 30 μL of mouse peripheral blood and allowed to react at 37° C. for 1 hour, and with a negative control, a low temperature (4° C.) treatment was applied to inhibit phagocytic response. After the phagocytic response, an average value of 10 times measured fluorescence (for 5 seconds) using the phagocyte phagocytic capacity evaluation apparatus was obtained and a measured value of the negative control was subtracted to determine a value of fluorescent intensity difference as a value of phagocytosis. Respective measured values were converted (normalized) such that the average value becomes 0 and the standard deviation becomes 1.

[Water Maze Test]

(1) Apparatus

A commercially available black ink was added to water (23±1° C.) in a circular cylindrical pool (diameter: 100 cm; depth: 40 cm) such that a swimming mouse cannot visually recognize a platform. Also, the transparent platform (diameter: 10 cm) was installed such as to be positioned 1 cm below the water surface. A video recording of swimming of each mouse was made with a commercially available digital camera installed directly above the pool water surface. Swimming paths were analyzed using an image analysis software, AnimalTracker, and in accordance with a method described in “Neuroinformatics, 14, 479-481, 2016.”

(2) Procedures

On the day before the test, each mouse was made to swim once to acclimate to the pool. As the procedure, each mouse was left for 20 seconds on the platform fixed 1 cm above the water surface and then made to swim freely for 30 seconds. Thereafter, the mouse was guided onto the platform with an experimenter's hand and left there for 20 seconds. Also, in placing a mouse into the pool, the mouse was made to enter the water facing the wall of the pool and the experimenter moved immediately to a position not visible from the mouse. On the first to fifth day, training was performed to make each mouse memorize the position of the platform (4 times/day). As the procedure of the training, each mouse was placed into the pool from an arbitrary position and made to swim for 60 seconds and search for the platform installed 1 cm below the water surface. The time required to reach the platform was recorded and if the platform could not be reached in 60 seconds, the time was recorded as 60 seconds. Also, a mouse that could not reach the platform in time was guided to the platform with the experimenter's hand. After reaching the platform, the mouse was left there for 20 seconds and then taken out from the pool. Also, although by the five days of training, reduction of the time required to reach the platform was seen in both groups, differences among the groups were not seen. A probe test was performed on the sixth day. For the probe test, the platform was removed from the pool, each mouse was made to swim for 60 seconds, and time spent in the quadrant of the pool in which the platform was present was measured. Also, the probe test was performed once on each mouse.

[Learning Function]

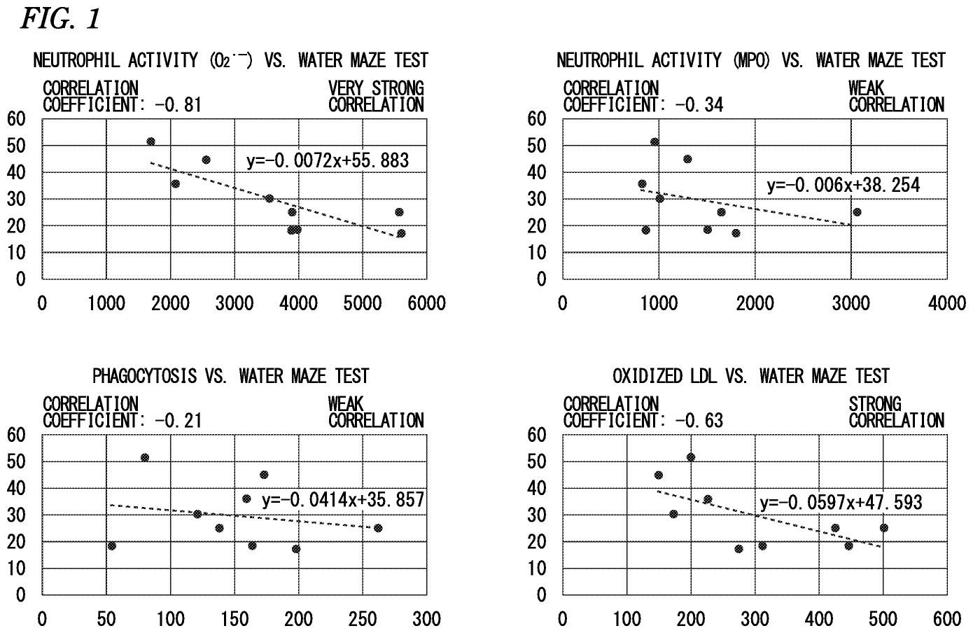

Statistical analysis was examined based on data for the Alzheimer's disease model mice (SAMP8). As a result of performing correlation analysis of the respective measured values of neutrophil activity, oxidized LDL, and phagocytosis and the conventional method for learning function evaluation (water maze test), a very strong correlation (correlation coefficient: −0.81) with the neutrophil activity (O₂^{⋅-} production activity) and a strong correlation (correlation coefficient: −0.63) with the oxidized LDL were seen (FIG. 1). The respective measured values of neutrophil activity, phagocytosis, and oxidized LDL were unified and whether or not prediction of learning function is possible was examined.

The respective measured values were converted such that the average value becomes 0 and the standard deviation becomes 1. A multiple regression analysis method was applied to the converted values (normalized values) and the following results were obtained.

(Water maze test)=−0.78×(O₂^{⋅-} production activity)−0.08×(oxidized LDL) Correlation coefficient=0.8228 (1)

(Water maze test)=−1.29×(O₂^{⋅-} production activity)+0.62×(MPO activity) Correlation coefficient=0.9131 (2)

(Water maze test)=−1.295×(O₂^{⋅-} production activity)+0.620×(MPO activity)+0.021×(phagocytosis) Correlation coefficient=0.9133 (3)

(Water maze test)=−1.264×(O₂^{⋅-} production activity)+0.787×(MPO activity)−0.316×(oxidized LDL) Correlation coefficient=0.9480 (4)

(Water maze test)=−1.24×(O₂^{⋅-} production activity)+0.79×(MPO activity)−0.05×(phagocytosis)−0.33×(oxidized LDL) Correlation coefficient=0.9489 (5)

The unified measured value exhibited a higher correlation in comparison to the individual measured values (unified: 0.9489; individual: −0.21 to −0.81) and significance of unifying the neutrophil activity, oxidized LDL, and phagocytosis was thus found.

Also, the results show that higher correlation coefficients are exhibited and it is thus more desirable when (O₂^{⋅-} production activity), (MPO activity), (oxidized LDL), and (phagocytosis) are used in the four-variable case, (O₂^{⋅-} production activity), (MPO activity), and (oxidized LDL) are used in the three-variable case, (O₂^{⋅-} production activity) and (MPO activity) are used in the two-variable case, and (O₂^{⋅-} production activity) is used in the single-variable case.

Among the above, it is notable that whereas among the individual variable cases, there is a strong correlation with the oxidized LDL (correlation coefficient: −0.63), among the two-variable cases, the combination of (O₂^{⋅-} production activity) and (MPO activity) showed a higher correlation coefficient (correlation coefficient=0.9131) than the combination of (O₂^{⋅-} production activity) and (oxidized LDL) (correlation coefficient=0.8228).

The respective measured values were converted such that the average value becomes 0 and the standard deviation becomes 1. The multiple regression analysis method was applied to the converted values (normalized values) and the following results were obtained.

Multiple Regression Analysis of Normalized Measured Values

(Order in which a Correlation Coefficient Higher than that of a Correlation Equation of an Individual Variable (Simple Regression Equation) is Obtained by Forming a Multiple Regression Equation)

TABLE 1

| | Correlation | |
|---|---|---|
| Multiple Regression Equation | Coefficient | * |
| (WMT) = −1.24(0₂•—) + 0.79(MPO) − 0.05(phagocytosis) + 0.33(oxLDL) | R = 0.949 | 0.129 |
| (WMT) = −1.26(0₂•—) + 0.78(MPO) + 0.32(oxLDL) | R = 0.948 | 0.128 |
| (WMT) = −0.80(HbA1c) − 0.44(phagocytosis) | R = 0.816 | 0.124 |
| (WMT) = −0.52(TG) − 0.60(0₂•—) | R = 0.944 | 0.124 |
| (WMT) = −0.14(MPO) − 0.38(oxLDL) | R = 0.469 | 0.111 |
| (WMT) = −1.30(0₂•—) + 0.62(MPO) − 0.02(phagocytosis) | R = 0.913 | 0.093 |
| (WMT) = −1.29(0₂•—) + 0.62(MPO) | R = 0.913 | 0.093 |
| (WMT) = −0.77(TG) − 0.37(MPO) | R = 0.854 | 0.089 |
| (WMT) = −0.05(MPO) − 0.29(phagocytosis) − 0.451(oxLDL) | R = 0.545 | 0.085 |
| (WMT) = −0.299(phagocytosis) − 0.479(oxLDL) | R = 0.540 | 0.084 |
| (WMT) = −0.38(HbA1c) − 0.63(0₂•—) | R = 0.886 | 0.066 |
| (WMT) = −0.33(MPO) − 0.21(phagocytosis) | R = 0.414 | 0.056 |
| (WMT) = −0.77(TG) − 0.27(phagocytosis) | R = 0.815 | 0.050 |
| (WMT) = −0.392(fasting BG) − 0.577(0₂•—) | R = 0.870 | 0.050 |
| (WMT) = −0.77(fasting BG) − 0.27(phagocytosis) | R = 0.811 | 0.046 |
| (WMT) = −0.64(HbA1c) − 0.20(MPO) | R = 0.718 | 0.035 |
| (WMT) = −0.64(TC) − 0.19(phagocytosis) | R = 0.692 | 0.027 |
| (WMT) = −0.575(TG) − 0.228(TC) − 0.042(HbA1c) | R = 0.789 | 0.024 |
| (WMT) = −0.203(TC) − 0.682(0₂•—) | R = 0.833 | 0.013 |
| (WMT) = −0.53(insulin) − 0.10(MPO) | R = 0.584 | 0.007 |
| (WMT) = −0.53(insulin) − 0.10(phagocytosis) | R = 0.584 | 0.007 |
| (WMT) = −0.68(fast BG) − 0.13(insulin) | R = 0.772 | 0.007 |
| (WMT) = −0.64(TC) − 0.055(MPO) | R = 0.670 | 0.005 |
| (WMT) = −0.76(0₂•—) − 0.05(phagocytosis) + 0.10(oxLDL) | R = 0.824 | 0.004 |
| (WMT) = −0.78(0₂•—) − 0.08(oxLDL) | R = 0.823 | 0.003 |
| (WMT) = −0.392(fasting BG) − 0.577(MPO) | R = 0.765 | 0 |
| (WMT) = −0.81(0₂•—) − 0.02(phagocytosis) | R = 0.820 | 0 |
| (WMT) = −0.03(insulin) − 0.84(0₂•—) | R = 0.820 | 0 |

WMT: Water maze test
*: Difference between the highest value of the correlation coefficients with respect to (Water maze test) exhibited individually by the respective items constituting the multiple regression equation and the correlation coefficient obtained by forming the multiple regression equation.

Simple Regression Analysis of Normalized Measured Values

(Order in which a High Correlation Coefficient is Exhibited by a Simple Regression Equation)

TABLE 2

| | Correlation |
|---|---|
| Simple Regression Equation | Coefficient |
| (WMT) = −0.820(0₂•—) | R = 0.820 |
| (WMT) = −0.769(TG) | R = 0.769 |
| (WMT) = −0.765(fasting BG) | R = 0.765 |
| (WMT) = −0.765(TC) | R = 0.765 |
| (WMT) = −0.692(HbA1c) | R = 0.692 |
| (WMT) = −0.577(insulin) | R = 0.577 |
| (WMT) = −0.455(oxLDL) | R = 0.455 |
| (WMT) = −0.358(MPO) | R = 0.358 |
| (WMT) = −0.261(phagocytosis) | R = 0.261 |

WMT: Water maze test

From the fact the correlation coefficients obtained by the multiple regression equations are higher than the correlation coefficients of the simple regression equations, it can be said that by multiple regression, equations are made to predict the water maze test (cognitive function) more accurately. It is thus shown that evaluating a selected plurality of items is useful for improved evaluation of cognitive function. Based on these points, it is most useful to measure the four items (O₂^{⋅-}, MPO, phagocytosis, and oxLDL) with which the correlation function became as high as 0.129 higher than with simple regression as shown in Table 1. Besides this, the combinations (O₂^{⋅-}, MPO, oxLDL), (HbA1c, phagocytosis), (TG, O₂^{⋅-}), and (MPO, oxLDL) that increased by 0.1 or more follow in being promising. Also, the combinations (O₂^{⋅-}, MPO, phagocytosis), (O₂^{⋅-}, MPO), (TG, MPO), (MPO, phagocytosis, oxLDL), (phagocytosis, oxLDL), (HbA1c, O₂^{⋅-}), (MPO, phagocytosis), (TG, phagocytosis), and (fasting BG, O₂^{⋅-}) that increased by 0.05 or more are also useful.

All publications, patents, and patent applications cited in the present description are incorporated in entirety as reference herein.

Also, the disclosure, including the description, claims, and drawings, of Japanese Patent Application No. 2017-173037 filed on Sep. 8, 2017 is herein incorporated in entirety as reference.

1. An Alzheimer's disease diagnosis device comprising:
a measurement means configured to measure one index or more selected, wherein at least superoxide production activity is selected, from the group consisting of superoxide production activity, myeloperoxidase activity, oxidized LDL level, phagocytosis, triglycerides, fasting blood glucose, total cholesterol, hemoglobin A1c, and insulin in peripheral blood; and
a displaying means configured to display an index measured by the measurement means as a pathological index for Alzheimer's disease.

2. The Alzheimer's disease diagnosis device according to claim 1, wherein the measurement means measures two indices or more selected from the group consisting of superoxide production activity, myeloperoxidase activity, oxidized LDL level, and phagocytosis and with which superoxide production activity is selected.

3. The Alzheimer's disease diagnosis device according to claim 1, wherein the measurement means measures superoxide production activity, myeloperoxidase activity, and oxidized LDL level.

4. An Alzheimer's disease diagnosis device comprising:
a measurement means configured to measure superoxide production activity, myeloperoxidase activity, oxidized LDL level, and phagocytosis in peripheral blood; and
a displaying means configured to display a×A+b×B+c×C+d×D with respect to the indices measured by the measurement means as a pathological index for Alzheimer's disease.
wherein,
A: normalized superoxide production activity
B: normalized myeloperoxidase activity
C: normalized oxidized LDL level
D: normalized phagocytosis
a, b, c, d: coefficients

5. A method, wherein one index or more selected, wherein at least superoxide production activity is selected, from the group consisting of superoxide production activity, myeloperoxidase activity, oxidized LDL level, phagocytosis, triglycerides, fasting blood glucose, total cholesterol, hemoglobin A1c, and insulin in collected peripheral blood is used as a pathological index for Alzheimer's disease.
